(19) United States
(12) Patent Application Publication
(10) Pub. No.: US 2007/0167920 A1
(43) Pub. Date: Jul. 19, 2007

(54) Automatic Injector

(76) Inventor: Edgar Hommann, Grossaffoltern (CH)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/673,272

(22) Filed: Feb. 9, 2007

Related U.S. Application Data
(63) Continuation of application No. PCT/CH05/00277, filed on May 18, 2005.

(30) Foreign Application Priority Data
Aug. 11, 2004 (DE) 10 2004 039 020.7

(51) Int. Cl. A61M 5/24; A61M 5/28

(52) U.S. Cl. 604/206

(57) ABSTRACT

An automatic injection device including a housing with a reservoir for an injectable product or substance, an injection needle connected to the reservoir, wherein the reservoir can be moved to move the injection needle relative to the housing, an injection drive for causing the movement of the injection needle, a dispensing drive including a conveyor element for dispensing the product and a motor for driving the conveyor element.

### CROSS-REFERENCE TO RELATED APPLICATION(S)

This application is a Continuation of International Application No. PCT/CH2005/000277, filed on May 18, 2005, which claims priority to German Application No. 10 2004 039 020.7, filed on Aug. 11, 2004, the contents of both of which are incorporated in their entirety by reference herein.

### BACKGROUND

The present invention relates to devices for delivering, dispensing, injecting or administering substances, and to methods of making and using such devices. More particularly, it relates to automatic injection devices and, in one embodiment, to an automatic injector for administering an injectable substance or product such as a medicament in liquid form, for example insulin, a growth hormone, an osteoporosis preparation, etc.

Automatic injectors are injection devices wherein, typically, when triggered, the injection needle automatically moves in an insertion or injection movement and the product or substance to be administered is automatically dispensed. Due to the movements that have to be performed to achieve this, such injection devices are typically complex. Some involve a large amount of filigree work and are therefore expensive and may be susceptible to operating and/or structural faults or weaknesses. In some cases, the operational movements of the devices and, during use, the injection, insertion or forward movement of the needle and dispensing of the product are operated by a single mechanical spring or an injection spring and a separate dispensing spring.

### SUMMARY

An object of the present invention is to provide an automatic injector or injection device which is simple to construct and dispenses the product to be administered in a uniform volume and/or flow during the administering or injecting operation.

In one embodiment, the present invention comprises an automatic injection device comprising a housing, a reservoir for an injectable substance, an injection needle carried by the reservoir, wherein the reservoir is movable to move the injection needle relative to the housing, an injection drive for causing the movement of the reservoir, and a dispensing drive comprising a conveyor for acting on the substance and a motor for driving the conveyor.

In some embodiments, the present invention relates to an automatic injector comprising a housing with a reservoir for an injectable product, an injection needle connected to the reservoir, an injection drive and a dispensing drive acting on the product to dispense it. The housing may serve as the reservoir itself. In some preferred embodiments, however, the reservoir is provided in the form of a container, e.g. an ampoule, which is accommodated in or can be accommodated in the housing. The conveying element may be a translating plunger, the movement of which forces the product out of the reservoir and by which the product is dispensed through the injection needle connected to the reservoir. However, the dispensing mechanism may also be provided in the form of a rotary pump or peristaltic pump. In principle, any type of positive-displacement pump or flow mechanism may be used for the dispensing mechanism. The injection drive is coupled with the injection needle so that it is able to cause an injection movement of the injection needle relative to the housing in a forward drive direction (which also may be thought of as the injection or insertion direction or movement). In use, the automatic injector is oriented towards the skin of a human or animal or positioned in readiness for administering the product and, after being triggered or actuated, driven by the injection drive, the injection needle moves forward in its injection movement into or through the skin as far as the subcutaneous tissue layers or deeper.

In some embodiments of the present invention, the product or substance to be delivered is dispensed on a motorized basis. To this end, the dispensing drive has a conveying element acting on the product and a motor, which drives the conveying element. The motor is provided in addition to the injection drive. In some preferred embodiments, the motor may be a rotary motor, more preferably an electric motor such as an electric stepper motor. In some preferred embodiments, the drive coupling operably connecting the motor and the conveying element is a mechanical coupling, e.g., a gear mechanism, which converts, e.g., reduces, a driving motion of the motor into a conveying motion of the conveying element.

Since the conveying movement of the conveying element and hence dispensing of the product are operated on a motorized basis, the product is administered uniformly, e.g., in a constant volume flow, per dispensing operation whenever the automatic injector is triggered. If using a dispensing spring, on the other hand, the spring force of the spring varies due to the fact that the spring has to be released to provide the driving motion, as a result of which the volume flow of the product, i.e. the quantity of product dispensed per unit of time, decreases during the course of the dispensing operation. If desirable however, the motor constituting the dispensing drive in accordance with the present invention may be controlled accordingly by a motor control system and/or also regulated if necessary. If desirable, the volume flow may be increased by the motor during the course of the dispensing operation. Generally speaking, however, in some embodiments, a constant volume flow is desirable. A motorized drive in accordance with the present invention may also be advantageous if administering highly viscous fluids, liquids or substances.

In view of the fact that a motor is used for dispensing purposes, the injection and dispensing mechanism can also be simplified, at least compared with automatic injectors of the type which have a separate spring drive for injecting and dispensing. Compared with automatic injectors with an injection and dispensing spring, which activate the injection by the injection needle and dispensing of the product, at least the sequence of injection and dispensing can be improved in addition to rendering the volume or volumetric flow more uniform, because these two phases can be separated more cleanly and reliably from one another.

In some embodiments of the present invention, when administering a product or substance, the operational sequence is such that, during a first phase, the injection needle is moved forward (i.e., in the injection or insertion movement) and the motor does not act on the conveying element until after completion of the needle movement or until administering the product after the injection movement. In some embodiments, a switch may be provided for the purpose of this sequential procedure, which switches the motor on at a selected time, e.g., after or as soon as the pre-injection or injection movement has been fully effected. If dispensing should be started during the pre-injection or injection movement of the injection needle, this can also be achieved by a switch which switches the motor on when the injection needle has reached a specific position in its movement during the course of its pre-injection or injection movement.

In some embodiments, the driving force used to drive the injection needle in its injection, insertion or forward movement is advantageously an elastic force.

In some preferred embodiments, the injection drive can be elastically biased opposite the forward drive direction. The injection drive may advantageously be biased opposite the forward drive direction by the motor. In such embodiments, the injection drive again can be biased after the injection needle has been driven forward by the motor, i.e. charged. Thus, in some embodiments, an automatic injector in accordance with the present invention can be used to administer product again and/or facilitates the disposal of an empty product reservoir.

In some preferred embodiments, the dispensing drive has an output member, by which the motor is coupled with the conveying element, by the output element being in an operable engagement, e.g., coupled or connected, with the motor, either directly or via one or more coupling members. A stop may be provided, against which the output member or a coupling member in the coupling train can be moved in a drive direction to or into a stop position. Once the output member or the relevant coupling member has reached the stop position, the conveying element can not be driven any further. However, if the motor continues driving in the drive direction, it is supported via the output member or the coupling member abutting with the stop instead of it, so that the ongoing motor drive or operation causes a part of the dispensing drive, preferably the motor, to move in the direction opposite the output movement of the output member, by which the injection drive can be biased opposite the forward drive direction.

In some preferred embodiments, the coupling engagement resulting in the coupling between the motor and output member is a thread engagement with a thread axis advantageously extending in the forward drive direction. The coupling engagement may also be provided as a thread engagement if the automatic injector does not have the feature whereby the injection drive can be biased.

In some preferred embodiments, to generate the force needed to produce the forward movement and effect the injection, the injection drive has a mechanical injection spring. Alternatively, the force may be generated by a compressed gas reservoir or a gas generator, for example a gas cartridge. A mechanical injection spring or several such springs may also be used in combination with compressed gas.

In some preferred embodiments, the motor is accommodated in a bearing structure, which can be moved in the forward drive direction by the injection drive. The injection drive causes the forward driving movement of the injection needle via the bearing structure. In an initial state, the bearing structure is in a proximal or rear position retained in engagement with the housing against the force of the injection drive in some embodiments, the bearing structure also provides a mount for a power source for the motor. The automatic injector has a trigger element, which releases the retaining engagement when activated.

In some preferred embodiments, to enable it to be driven forward, the injection needle is secured to the reservoir or a reservoir holder accommodating the reservoir, e.g. in the form of a container, which supports it in the forward drive direction. To drive the injection needle forward, the bearing structure acts on the reservoir or reservoir holder. To this end, it pushes the reservoir or the reservoir holder in the forward drive direction and can be moved away from the reservoir holder opposite the forward drive direction, i.e. it advantageously acts on the reservoir or the reservoir holder or an intermediate structure by a pure pressure contact. It is advantageous to use a reservoir holder if the reservoir is provided in the form of a standard container, such as an ampoule or glass ampoule closed by a plunger. In a design of this type, the reservoir is advantageously not required to absorb the force of the injection drive.

### BRIEF DESCRIPTION OF THE DRAWINGS

FIG. 1 shows an embodiment of an automatic injector or injection device in accordance with the present invention in an initial state, in which the injection needle of the injector has assumed a distal or forward position, and

FIGS. 2-7 show the embodiment of FIG. 1 in a sequence of states which the injector assumes as a product or substance is being administered.

### DETAILED DESCRIPTION

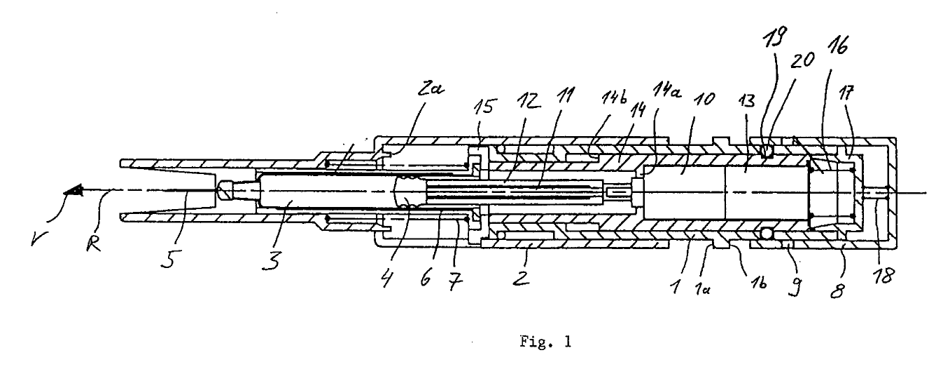

FIG. 1 is a longitudinal section illustrating one embodiment of an automatic injector in accordance with the present invention. The automatic injector is in an initial state prior to administering a product or substance to be injected. In the initial state, the automatic injector may be stored or manipulated until the product has been directly administered, for example. Overall, the shape of the automatic injector is slim and compact, e.g., in the form of injection devices known as injection pens.

The automatic injector has a housing comprising a sleeve-shaped proximal or rear housing portion 1 and a sleeve-shaped distal or forward housing portion 2, which are coupled or connected to one another so that they are able to move relative to one another along a common central axis of symmetry R. During the axial movement, the housing portions 1 and 2 are linearly guided on one another along the axis R.

A reservoir 3 filled with the product to be injected or administered is mounted in the distal housing portion 2 so that it can be displaced along the axis R in a forward drive direction V and opposite the forward drive direction V, during which the distal housing portion 2 acts as a linear guide for the movement of the reservoir 3. In the embodiment illustrated as an example, the reservoir 3 is provided in the form of a container, e.g., a standard ampoule. Attached to the end of the reservoir 3 is an injection needle 5, which projects in the forward drive direction V. A plunger 4 is accommodated in the reservoir 3 so that it can be displaced along the axis R. The plunger 4 closes off the reservoir 3, rendering it product-tight. Due to a movement of the plunger 4 in the forward drive direction V, product is forced out of the reservoir and dispensed through the hollow injection needle 5. However, the injection needle 5 need not necessarily be hollow. The product may also be directed around the external periphery of the injection needle. The axis R along which both the reservoir 3 can be moved relative to the housing portion 2 and the plunger 4 can be moved relative to the reservoir 3, may be referred to hereafter as the forward drive axis. The reservoir 3 is accommodated in a reservoir holder 6, which reservoir holder 6 positions or centers the reservoir 3, and the reservoir 3 abuts against a stop of the reservoir holder 6 in the forward drive direction V. The housing portion 2 directly guides the reservoir holder 6 linearly in the axial direction.

A return spring 7 distally supported against the housing portion 1 and proximally supported on the reservoir holder 6 biases the reservoir holder 6 relative to the housing portion 2 opposite the forward drive direction V against a stop, which is axially fixed on housing portion 1 in the initial state.

In the initial state, housing portion 2 assumes its farthest position relative to housing portion 1 in the distal direction, in which it forms a needle guard for the injection needle 5 in conjunction with a distal sleeve portion, which surrounds the injection needle 5 and projects beyond its tip in the forward drive direction V. From this farthest position in the distal direction, housing portion 2 can be moved against the force of the return spring 7 relative to housing portion 1 opposite the forward drive direction V to a position against a stop 1a of the housing portion 1.

The proximal housing portion 1 mounts, carries or contains a drive mechanism comprising an injection drive and a dispensing drive. The injection drive drives the injection needle 5 forward to enable it to pierce organic tissue, e.g. to move into and/or through human skin. The dispensing drive acts independently of the injection drive and when the injection needle 5 has completed the forward drive movement dispenses the product from the reservoir 3 through the injection needle 5.

The dispensing drive comprises an electric stepper motor 10 as a drive or power unit. An electric battery 13 is provided as a power source for the motor 10. By its motor shaft 11, the motor 10 acts on an output member 12 forming a plunger rod for the plunger 4. The motor shaft 11 and output member 12 are coupled with one another in an operable coupling engagement, which is provided in the form of a threaded engagement in the embodiment illustrated as an example. To this end, the output member 12 is provided with an internal thread which constitutes the thread engagement or coupling engagement with the external thread of the motor shaft 11. The forward drive axis R is simultaneously the rotation axis of the motor shaft 11 and, in the coupling engagement, the thread axis of the two threads of the motor shaft 11 and output member 12. The output member 12 sits in abutment against the plunger 4 in the forward drive direction V, i.e., in the depicted embodiment, there is only a pressure contact between the plunger 4 and the output member 12 but no fixed connection.

The drive mechanism also comprises a bearing structure 14 which supports the motor 10 in the forward drive direction V and, in the illustrated exemplary embodiment, does so by a stop 14a formed by an inner shoulder of the bearing structure 14. The bearing structure 14 also provides a mount for the motor 10 so that the motor 10, i.e. the stator part of the motor 10, can not rotate about the forward drive axis V relative to the bearing structure 14. The bearing structure 14 is a hollow sleeve, the hollow cross-section of which divides the shoulder 14a into a proximal and a distal sleeve portion. The bearing structure 14 is mounted in the proximal housing portion 1 so that it can be moved backwards and forwards along the forward drive axis V and is linearly guided in the axial direction by the housing portion 1 and prevented from rotating with respect to the axis R.

The injection drive has an injection spring 16 as a force generator. The injection spring 16 acts on the bearing structure 14 in the forward drive direction V. It is proximally secured to a cap 17 attached to the proximal end of the housing portion 1 and distally supported on the motor 10 via the battery 13. The injection spring 16 therefore acts via the motor 10 and its axial support on the stop 14a in the direction towards the bearing structure 14.

In the initial state, the proximal housing portion 1 and the bearing structure 14 are in a retaining engagement, which prevents the bearing structure 14 from moving in the forward drive direction V relative to housing portion 1 and holds the injection spring 16 biased or in a charged state. The retaining engagement is achieved by blocking or locking elements 20, for example balls or spherical pins. The blocking elements 20 are partially accommodated in a recess 19 of the bearing structure 14, i.e. one recess 19 respectively for each of the blocking elements 20. The recesses 19 are formed by indentations in the external surface of the bearing structure 14. Instead of individual recesses 19, it would also be possible to provide a common recess for all the blocking elements 20, for example in the form of a circumferentially extending groove in the external surface of the bearing structure 14. Radially opposite the recesses 19, the housing portion 1 is provided with orifices into which the blocking elements 20 project. Elastic forces act radially outwards on the blocking elements 20. However, a trigger element 8 prevents the blocking elements 20 from being able to move out of the recesses 19 under the effect of the elastic forces. The trigger element 8 comprises a sleeve portion and a base closing off the sleeve portion at the proximal end. By the sleeve portion, the trigger element 8 surrounds the orifices of the housing portion 1 and thus holds the blocking elements 20 in the recesses 19 of the bearing structure 14. A spring 18 supported on the housing portion 1 and the trigger element 8 biases or urges the trigger element 8 relative to the housing 1 into a proximal position. The trigger element 8 can be pushed against the elastic force of the spring 18 relative to the housing portion 1 in the forward drive direction V as far as a stop 1b formed by the housing portion 1. The stop 1b and also the stop 1a for housing portion 2 are formed by a circumferentially extending shoulder on the external surface of the housing portion 1.

With respect to the bearing structure 14, it should also be pointed out that it forms a stop 14b on its external surface and, in the embodiment illustrated as an example, does so in the form of a circumferentially extending stepped edge. The housing portion 1 acts as a counter-stop with respect to the stop 14b opposite the forward drive direction V.

Disposed axially between the bearing structure 14 and the reservoir holder 6 is a guide structure 15, by which the bearing structure 14 acts on the reservoir holder 6 in the forward drive direction V. The distal housing portion 2 guides the guide structure 15 linearly in the axial direction, preventing it from rotating, i.e. the guide structure 15 is able to move only in and opposite to the forward drive direction V relative to the housing portion 2. Between the guide structure 15 and the bearing structure 14 on the one hand and between the guide structure 15 and the reservoir holder 6 on the other hand, there is nothing more than an axial pressure contact. The guide structure 15 guides the output member 12 linearly in the axial direction, preventing it from rotating, so that a rotating movement of the motor 10 causes an axial movement of the output member 12 via the coupling engagement of the motor shaft 11 and the output member 12.

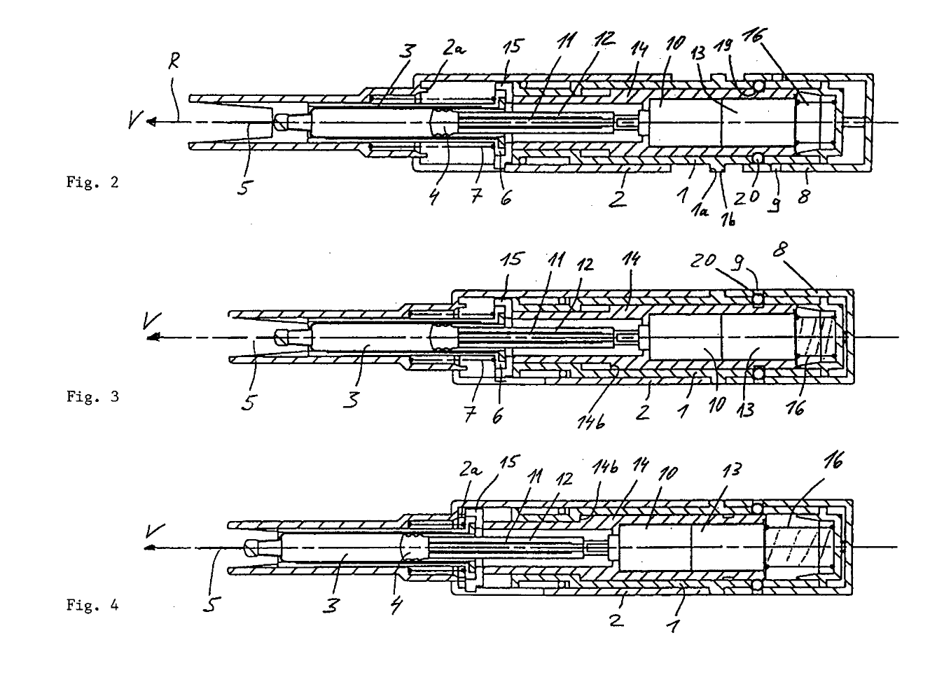

The operation and/or use of the exemplary automatic injector will be explained below with reference to the sequences illustrated in FIGS. 2-7, which depict one embodiment of a sequence of an injection in accordance with the present invention.

FIG. 2 illustrates the automatic injector in an initial state and is identical to FIG. 1. The user places the automatic injector, in the initial state, against the desired injection point or site on the skin so that the needle guard formed by the housing portion 2 surrounds the injection point. When pressure is applied towards the injection point, housing portion 2 moves relative to housing portion 1, opposite the forward drive direction V. This movement as far as the stop 1a is effected against the elastic force of the return spring 7, which is supported on the housing portion 1 by the retaining engagement in the initial state. The path length of this movement is dimensioned by the stop 1a so that the tip of the injection needle 5 sits at a short distance before the skin surface, but has not yet come into contact with the skin surface. To release the retaining engagement of the bearing structure 14, the user pushes the trigger element 8 in the forward direction V as far as the stop 1b in a next step. In the stop position, recesses or orifices 9 in the sleeve portion of the trigger element 8 are moved so that they radially overlap with the blocking elements 20. As soon as the trigger element 8 assumes its stop position, the blocking elements 20 therefore move radially outwards into the recesses or orifices 9 due to the elastic forces acting on them and simultaneously move out of the recesses 19 of the bearing structure 14. The retaining engagement is now released.

FIG. 2 illustrates the automatic injector in a brief transition state in which the trigger element 8 has just reached its stop position and the retaining engagement has been released, but the bearing structure 14 has not yet started its forward drive movement. The injection spring 16 acting on the bearing structure 14 via the battery 13 and the motor 10 can now relax and drive the bearing structure 14 in the forward drive direction V. The bearing structure 14 is moved in the forward drive direction V via the guide structure 15 so that it abuts with the reservoir holder 6, i.e. the injection spring 16 acts on the reservoir holder 6 via the bearing structure 14 and drives it in the forward drive direction V. The return spring 7 is significantly weaker than the injection spring 16, but nevertheless opposes the forward driving action caused by the injection spring 16 to a negligible degree. Due to the forward driving action of the reservoir holder 6, the reservoir 3 and the injection needle 5 attached to it are likewise moved in the forward drive direction V until the bearing structure 14 reaches a position wherein its stop 14b is against the counter-stop of the housing portion 1. The forward drive movement therefore corresponds to a forward piercing or injection movement of the injection needle 5. The path length of the forward drive movement of the bearing structure 14 corresponds to the desired injection depth.

FIG. 4 illustrates the automatic injector after the forward piercing or injection movement has been completed. Immediately following the injection movement, the motor is switched on. The forward piercing or injection movement takes place separately in time from the operation of dispensing of the product, i.e. they take place sequentially one after the other.

After the injection movement is terminated, the guide structure 15 moves into a blocking engagement, e.g., with the distal housing portion 2, by which the guide structure 15 is retained in the distal position it has reached relative to the reservoir 3. The blocking engagement may be a positive connection. It prevents the guide structure 15 from moving in the proximal direction. Furthermore, when the injection movement is complete, the guide structure 15 is pushed by the bearing structure 14 in the distal direction against a contact 2a which may be provided in the form of the distal housing portion 2, as is the case with the illustrated exemplary embodiment. In this embodiment, a projection, for example a circumferentially extending annular web or, alternatively, a single cam projecting in the proximal direction, constitutes the contact 2a. The guide structure 15 is provided with a counter-contact. The contact 2a and the counter-contact constitute a signal transmitter, and in the illustrated embodiment, a switch, which is connected by a signal line to a control of the motor 10. When the contact is established between the contact 2a and the counter-contact of the guide structure 15, the signal transmitter signals to the control via the signal line that the injection movement has terminated. The control then switches on the motor, for example by connecting the motor 10 to the battery 13. Although the contact 2a and its counter-contact are used as a signal transmitter for the control, the contact 2a and the counter-contact may also form a switch and close the circuit for the power supply of the motor 10 directly, i.e. they may be a switch element of the circuit.

To serve as a signal transmitter, the contact 2a and its counter-contact do not necessarily have to form a contact switch. Accordingly, the contact 2a or its counter-contact may be replaced by a detector which operates without contact, for example.

Switched on, the motor 10 acts via the motor shaft 11 and coupling engagement on the output member 12, which is moved in the forward drive direction V due to its linear guide, driving the plunger 4 with it due to a pressure contact. The forward drive movement of the output member 12 is restricted by a stop afforded by the guide structure 15 or the reservoir holder 6, against which the output member 12 moves into a pressure contact once the reservoir 3 has been emptied. Alternatively, the forward drive movement could be stopped by the force acting on the motor shaft 11 due to the plunger 4 pushing against the reservoir 3 in the forward drive direction V.

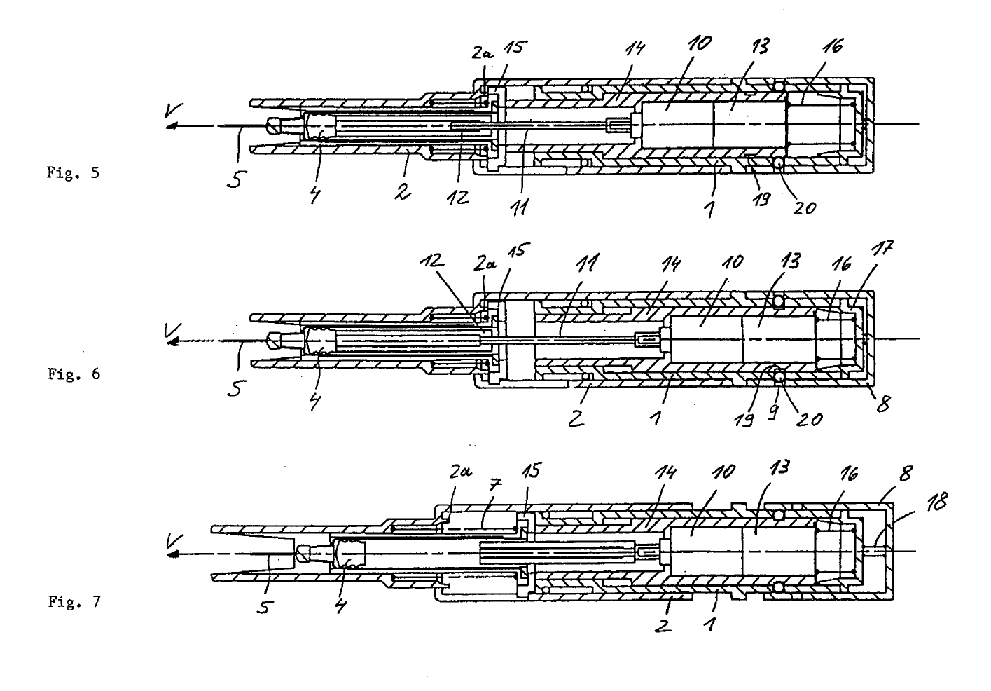

FIG. 5 illustrates the automatic injector after the reservoir 3 has emptied, i.e. with the output member 12 and/or plunger 4 disposed in a stop position. The motor 10 is still running in the same direction of rotation when the output member 12 and/or the plunger 4 is in the stop position. However, it is now supported via the output member 12 against its stop so that the motor 10 moves opposite the forward drive direction due to the coupling engagement with the output member 12 which is still prevented from rotating. With respect to this resetting movement, the bearing structure 14 is also connected to the motor 10 so that it can not move axially and the motor 10 therefore also drives it with it. The motor 10 is sufficiently strong to permit the resetting movement against the force of the biasing injection spring 16 until the recesses 19 of the bearing structure 14 reach the position radially overlapping with the orifices of the housing portion 1 again and the retaining engagement between the housing portion 1 and the bearing structure 14 can be re-established. The thread of the motor shaft 11 extends axially across at least the sum of the lengths of the injection movement or re-setting movement of the motor 10 and the maximum stroke of the output member 12.

FIG. 6 illustrates the automatic injector after the resetting movement has been completed, immediately before the retaining engagement is re-established. The resetting movement is also restricted or controlled by a stop of the housing portion 1, in this instance formed by the cap 17. The stop is positioned so that the orifices of the housing portion 1 and the blocking elements 20 radially guided in them overlap radially with the recesses 19 of the bearing structure 14. However, the blocking elements 20 are still biased or urged outwardly into the recesses or orifices 9 of the trigger element 8 due to the elastic forces acting on them. However, until the user releases the pressure on the trigger element 8, the spring 18 pushes the trigger element 8 relative to the housing portion 1 opposite the forward drive direction V. Accordingly, it slides past the blocking elements 20 projecting into its recesses or orifices 9 and thus pushes them radially inwards into the recesses 19. The retaining engagement is therefore re-established so that the bearing structure 14 is positively locked in its proximal position with the housing portion 1 again against the force of the injection spring 16.

When the automatic injector in the state illustrated in FIG. 6 is removed from the injection point after an injection is performed, the housing portion 2 pushes back in the distal direction relative to the housing portion 1, again due to the rebounding elastic force of the return spring 7, until it forms a needle guard again. As a result of the movement in the distal direction, the blocking engagement of the guide structure 15 is released and the return spring 7 pushes the guide structure 15 back in the distal direction until it sits against the bearing structure 14. The motor shaft 11, output member 12, bearing structure 14 and guide structure 15 now move into and/or assume the same positions they were in prior to the injection.

FIG. 7 illustrates the automatic injector after the injection. The distal housing portion 2 can be released from the proximal housing portion 1 and the reservoir 3 can now be replaced with a new one or simply disposed of.

Embodiments of the present invention, including preferred embodiments, have been presented for the purpose of illustration and description. They are not intended to be exhaustive or to limit the invention to the precise forms and steps disclosed. The embodiments were chosen and described to provide the best illustration of the principles of the invention and the practical application thereof, and to enable one of ordinary skill in the art to utilize the invention in various embodiments and with various modifications as are suited to the particular use contemplated. All such modifications and variations are within the scope of the invention as determined by the appended claims when interpreted in accordance with the breadth they are fairly, legally, and equitably entitled.

1. An automatic injection device comprising a housing, a reservoir for an injectable substance, an injection needle carried by the reservoir, wherein the reservoir is movable to move the injection needle relative to the housing, an injection drive for causing the movement of the reservoir, and a dispensing drive comprising a conveyor for acting on the substance and a motor for driving the conveyor.

2. The automatic injection device as claimed in claim 1, wherein the motor is switched on when the movement of the reservoir has stopped.

3. The automatic injection device as in claim 2, wherein the conveying element comprises a moveable plunger in the reservoir for dispensing the product and a gear mechanism operably coupling the motor and the plunger.

4. An automatic injector comprising:
a) a housing comprising a reservoir for an injectable product,
b) an injection needle connected to the reservoir, said reservoir moveable in a forward drive direction relative to the housing to effect an injection movement of the needle,
c) an injection drive for moving the reservoir, and
d) a dispensing drive comprising a conveying element associated with the reservoir and acting on the product to dispense the product and a motor for acting on the conveying element.

5. An automatic injector comprising:
a) a housing with a reservoir for an injectable product,
b) an injection needle connected to the reservoir, which reservoir can be moved in a forward drive direction relative to the housing to effect an injection movement,
c) an injection drive for causing the injection movement, and
d) a dispensing drive with a conveying element acting on the product to dispense the product and a motor for the conveying element provided in addition to the injection drive.

6. The automatic injector as claimed in claim 5, wherein the injection drive also moves the motor in the forward drive direction during the injection movement and is elastically biased against the forward drive direction by the motor.

7. The automatic injector as claimed in claim 5, wherein the dispensing drive comprises an output member via which the motor is coupled with the conveying element in a coupling engagement so that the output member can be moved by the motor in the forward drive direction as far as a stop position, and wherein in the stop position the injection drive is biased against the forward drive direction by the motor.

8. The automatic injector as claimed in claim 7, wherein, in the stop position, the motor can be moved opposite the forward drive direction by its own motor force in the coupling engagement.

9. The automatic injector as claimed in claim 8, wherein the coupling engagement is a thread engagement.

10. The automatic injector as claimed in claim 8, wherein the output member can be moved in the forward drive direction in the coupling engagement by the motor.

11. The automatic injector as claimed in claim 8, further comprising a stop against which the output member can be moved into the stop position.

12. The automatic injector as claimed in claim 11, wherein the output member acts directly on the conveying element by pressure contact in the forward drive direction.

13. The automatic injector as claimed in claim 5, wherein the injection drive has a force generator which acts on the motor directly or via one or more intermediate members during the injection movement.

14. The automatic injector as claimed in claim 5, wherein the injection drive comprises an injection spring which generates a spring force causing the injection movement.

15. The automatic injector as claimed in claim 5, wherein the motor is switched on when the injection movement has terminated.

16. The automatic injector as claimed in claim 5, further comprising a position detector for detecting a position of the injection drive, wherein the motor is switched on by the position detector.

17. The automatic injector according to claim 16, wherein the motor is switched on after the injection movement has terminated.

18. The automatic injector as claimed in claim 16, wherein the position detector comprises one of a switch contact and a counter-contact which act as a signal transmitter activating a control of the motor or an interrupter switch in a power supply circuit associated with the motor.

19. The automatic injector as claimed in claim 5, wherein the conveying element comprises a plunger accommodated in the reservoir so that it can be moved in the forward drive direction for dispensing the product and a gear mechanism, the plunger being operably coupled with the motor via the gear mechanism.

20. The automatic injector as claimed in claim 19, wherein the dispensing drive comprises an output member via which the motor is coupled with the conveying element in a coupling engagement so that the output member can be moved by the motor in the forward drive direction as far as a stop position, and wherein in the stop position the injection drive is biased against the forward drive direction by the motor, and wherein the output member forms a plunger rod and pushes against the conveying element in the forward drive direction and can be moved away from the conveying element opposite the forward drive direction.

21. The automatic injector as claimed in claim 20, wherein the motor is a rotary motor.

22. The automatic injector as claimed in claim 20, wherein the motor is an electric motor.

23. The automatic injector as claimed in claim 20, wherein the motor comprises a motor shaft forming, with the output member, part of the coupling engagement.

24. The automatic injector as claimed in claim 23, wherein the motor shaft has a rotation axis aligned with the forward drive direction.

25. The automatic injector as claimed in claim 5, wherein the injection drive drives the dispensing drive, the reservoir and the injection needle jointly in the forward drive direction during the injection movement.

26. The automatic injector as claimed in claim 5, further comprising a return spring which causes a retraction movement of the injection needle opposite the forward drive direction.

27. The automatic injector as claimed in claim 5, wherein the injection drive comprises a bearing structure and a force generator, and the bearing structure provides a mounting support for the motor in the forward drive direction and can be moved relative to the housing in the forward drive direction by the force generator.

28. The automatic injector as claimed in claim 27, wherein the injection drive causes the injection movement of the injection needle via the bearing structure.

29. The automatic injector as claimed in claim 5, wherein the dispensing drive comprises an output member by which the motor drives the conveying element, and the automatic injector further comprises a guide structure which guides the output member in the forward drive direction, wherein the motor can be moved opposite the forward drive direction.

30. The automatic injector as claimed in claim 29, wherein the guide structure is retained in a blocking engagement in a distal position when the motor is moving opposite the forward drive direction.

31. The automatic injector as claimed in claim 27, wherein the bearing structure is in a retaining engagement with the housing in a proximal position against the force of a force generator of the injection drive and the automatic injector has a trigger element which enables the retaining engagement to be released when activated.

32. The automatic injector as claimed in claim 31, wherein a container forms the reservoir and the automatic injector has a reservoir holder which accommodates the reservoir and which can be moved relative to the housing in the forward drive direction, and the injection needle is attached to the reservoir or the container holder.

33. The automatic injector as claimed in claim 32, wherein the bearing structure pushes the reservoir container in the forward drive direction and can be moved away from the reservoir holder opposite the forward drive direction.
